(19) United States
(12) Patent Application Publication
(10) Pub. No.: US 2009/0305238 A1
(43) Pub. Date: Dec. 10, 2009

(54) Microarray Microcard

(75) Inventor: David M. Cox, Foster City, CA (US)

Correspondence Address: KILYK & BOWERSOX, P.L.L.C., 3925 CHAIN BRIDGE ROAD, SUITE D401, FAIRFAX, VA, 22030, US

(73) Assignee: Applera Corporation, Foster City, CA (US)

(21) Appl. No.: 11/921,333

(22) PCT Filed: Jan. 22, 2007
(86) PCT No.: PCT/US07/01573

§ 371 Date: Mar. 16, 2009

Related U.S. Application Data
(63) Continuation-in-part of application No. 11/337,287, filed on Jan. 23, 2006, now abandoned.

(51) Int. Cl. C12Q 1/68; C12M 1/34

(52) U.S. Cl. 435/6; 435/287.2

(57) ABSTRACT

A fluid processing device is provided that comprises a substrate including a surface and a fluid processing pathway at least partially formed in or on the surface. The fluid processing pathway can comprise a channel, a reaction region in fluid communication with the channel, a microarray in fluid communication with the channel, and optionally a deformable valve. The microarray can comprise binding and/or detection sites, and each site can comprise a binding moiety. A method and a system using the fluid processing device, are also provided.

### CROSS-REFERENCE TO RELATED APPLICATIONS

The present application is a National Stage of PCT/US2007/001573, filed Jan. 22, 2007, which claims priority benefit from earlier filed U.S. patent application Ser. No. 11/337,287, filed Jan. 23, 2006, both of which are herein incorporated by reference in their entireties.

### INTRODUCTION

The present teachings relate to nucleic acid detection, ligation, amplification, and sequencing reactions and devices to carry out such reactions.

### SUMMARY

According to various embodiments, a fluid processing device is provided that comprises: a substrate comprising a surface; and a fluid processing pathway at least partially formed in or on the surface. The fluid processing pathway can comprise a channel, a reaction region in fluid communication with the channel, and a microarray in fluid communication with the channel. The microarray can comprise binding sites, each binding site comprising a binding moiety.

According to various embodiments, a method is provided that can comprise: providing a fluid processing device comprising a channel, a biological reagent disposed in a reaction region wherein the reaction region is in fluid communication with the channel, and a microarray in fluid communication with the channel; loading a sample into the reaction region; reacting the sample and the biological reagent to form a reaction product; and moving the reaction product from the reaction region through the channel and into the microarray. The microarray can comprise binding sites and each binding site can comprise a binding moiety.

According to various embodiments, a system is provided that can comprise: a platen; a fluid processing device holder disposed in the platen and adapted to hold at least one fluid processing device; at least one fluid processing device comprising a microarray, the microarray comprising binding sites; and a detector adapted to detect a detectable characteristic from at least one of the binding sites. The fluid processing device can comprise a substrate that can include a surface and a fluid processing pathway at least partially formed in or on the surface. The fluid processing pathway can comprise a channel, a reaction region in fluid communication with the channel, and a microarray in fluid communication with the channel. The microarray can comprise a plurality of the binding sites attached to a solid surface and each binding site can comprise at least a binding moiety. The microarray can comprise a plurality of different binding moieties.

Additional features and advantages of the present teachings will be set forth in part in the description that follows, and in part will be apparent from the description, or can be learned by practice of the present teachings.

### BRIEF DESCRIPTION OF THE DRAWINGS

FIG. 1 is a top plan view of a fluid processing device according to various embodiments;

FIG. 2 is a partial side cross-sectional view of the device taken along line 2-2 of FIG. 1;

FIG. 3 illustrates an embodiment of a fluidic device

FIG. 4 is an enlarged, perspective view of a fluid processing device according to various embodiments; and

FIG. 5 is an enlarged, perspective view of a fluid processing device according to various embodiments.

It is to be understood that both the foregoing general description and the following detailed description are exemplary and explanatory only.

### DESCRIPTION OF VARIOUS EMBODIMENTS

The term “reagent,” should be understood to comprise any reaction component that in any way affects how a desired reaction can proceed or be analyzed. The reagent can comprise a reactive or non-reactive component. It is not necessary for the reagent to participate in the reaction. The reagent can be a recoverable component comprising for example, a solvent and/or a catalyst. The reagent can comprise a promoter, accelerant, or retardant that is not necessary for a reaction but affects the reaction, for example, affects the rate of the reaction. The term “reagent” is used synonymous with the term “reaction component.” Analyses using a “reagent” can comprise reactions such as a nucleic acid amplification reaction, a polymerase chain reaction (PCR), a ligase chain reaction, an antibody binding reaction, an oligonucleotide ligation assay, a hybridization assay, or the like. Examples of reagents can comprise, for example, nucleic acids, primers, probes, polypeptides, a member of a binding pair, a buffer, or a nucleic acid sequence that hybridizes to another nucleic acid sequence.

“Nucleic acid” as used herein refers to nucleotides, oligonucleotides, DNA, RNA, PNA, etc. as these terms are understood by those skilled in the art. A DNA sequence hybridizing or binding to another sequence can also be said to react with that sequence.

According to various embodiments, devices and method are provided that can be used for sample preparation and microarray reactions on the same microcard. Examples of such reactions can comprise DNA or RNA amplification, labeling and a microarray reaction on the same microcard. In various embodiments, the microcard can be read on the same system that does the sample preparation. A sample preparation or reaction can be performed in a reaction region and can then be flowed to a microarray, both of which are located on a single microcard.

According to various embodiments, a fluid processing device is provided that comprises a substrate comprising a surface, and a fluid processing pathway at least partially formed in or on the surface. The fluid processing pathway can comprise a channel, a reaction region in fluid communication with the channel, and a microarray in fluid communication with the channel. The microarray can comprise binding sites, with each binding site comprising a binding moiety, for example, an immobilized binding moiety configured to bind with a target analyte. The binding can comprise, for example, binding due to hydrogen bonding of complementary nucleic acid sequences. The complementary nucleic acid sequences can comprise labels.

The sample components, analytes to be measured, or detectable moieties, can be labeled to facilitate sensitive and accurate detection. Labels may be direct labels which themselves are detectable or indirect labels which are detectable in combination with other agents. Exemplary direct labels include but are not limited to fluorophores, fluorescence resonance energy transfer (FRET) labels, chromophores, spin-labels, chemiluminescent labels, dioxetane-producing moieties, radioisotopes, and the like. Detectable moieties or analytes in a sample to be measured can be labeled to facilitate accurate and sensitive detection. Labels can be direct labels which themselves are detectable, or indirect labels which are detectable in combination with other agents. Exemplary direct labels include, for example, fluorophores, chromophores, spin-labels, chemiluminescent labels, dioxetane-producing moieties, radio isotopes, or nanoprobes. Exemplary indirect labels include enzymes which catalyze a signal-producing event, and ligands such as an antigen or biotin which can bind specifically with high affinity to a detectable anti-ligand, such as a labeled antibody or avidin. Many references on labeling molecules of interest, such as DNA, proteins, polysaccharides, and the like, are available.

According to various embodiments, the reaction region can have a volume of from about 0.001 μl to about 0.1 μl, from about 0.1 μl to about 10 μl, from about 1 μl to about 100 μl, from about 10 μl to about 100 μl, or from about 100 μl to about 5000 μl. The reaction region can comprise at least one reagent. In various embodiments, the at least one reagent can participate in a reaction comprising at least one of a nucleic acid amplification reaction, a purification reaction, a sequencing reaction, and a hybridization reaction. The reaction region can comprise a first reaction region, and a second reaction region in fluid communication with the first reaction region. At least one biological reagent for performing a nucleic acid amplification reaction can be disposed in the first reaction region. In various embodiments, at least one reagent can be disposed in the second reaction region and/or at least one reagent can be disposed in the microarray.

According to various embodiments, the binding moiety in the microarray can comprise a fluorophore. The fluorophore can be attached to or intercalated into an oligonucleotide. An oligonucleotide spot can comprise the binding moiety and an oligonucleotide, and a binding site can comprise an oligonucleotide spot. The oligonucleotide in an oligonucleotide spot can comprise at least one or a combination of a 5′-exonuclease probe, a stem-loop beacon probe, a zip-code sequence, and a stemless beacon probe. The oligonucleotide in an oligonucleotide spot can be complementary to a region of a target gene sequence of interest and thereby bind to that target sequence.

The binding sites can be spaced-apart from each other. The density of the binding sites can be at least one binding site per 100 μm², at least one binding site per 10 μm², or at least one binding site per 1 μm². Binding sites can be designed to detect genes or individual allelic variations thereof, or other sequences of interest. Each binding site can comprise a sequence that binds or reacts with a different allele.

According to various embodiments, the fluid processing pathway can comprise a valve disposed in the channel. The valve can be a deformable valve. The fluid processing pathway can comprise a plurality of fluid processing pathways. In various embodiments, each fluid processing pathway can comprise a respective reaction chamber and/or the microarray can comprise a plurality of microarrays, each microarray in fluid communication with the channel. The fluid processing device can comprise a fluid flow control device and can manipulate the fluid flow control device prior to moving the reaction product in the microarray.

According to various embodiments, the fluid processing device can comprise, for example, polyacrylamide, polypropylene, silicon, glass, plastic, or any combination thereof. The material can be an autoclavable material.

According to various embodiments, the fluid processing device can comprise a waste chamber in fluid communication with the microarray. The fluid processing device can comprise a thermal insulator disposed in or on the substrate and adapted to thermally insulate the reaction region from the microarray. The fluid processing device can comprise an identifier. The identifier can be a machine readable identifier.

According to various embodiments, a method is provided that can comprise: providing a fluid processing device comprising a channel, a reaction region in fluid communication with the channel, a reagent disposed in the reaction region, and a microarray in fluid communication with the channel; loading a sample into the reaction region; reacting the sample and biological reagent to form a reaction product; and moving the reaction product from the reaction region to the microarray. The microarray can comprise binding sites, and each binding site can comprise a binding moiety.

According to various embodiments, the method can comprise hybridizing the reaction product with a nucleic-acid. The nucleic-acid can comprise at least one DNA fragment, or any know nucleic acid sequence. The method can comprise detecting a detection characteristic at one or more of the binding sites. The detecting can comprise detecting luminescence or fluorescence emissions from at least one of the binding sites. The detecting can comprise illuminating at least one the binding sites with at least one excitation beam. In various embodiments, the method can comprise purifying the reaction product prior to filling the microarray.

The sample can comprise at least one nucleotide. In various embodiments, the sample can comprise a biological sample or analyte. The reaction product can comprise a nucleotide amplification reaction product or a sequencing reaction product. The method can comprise heating the reaction region to a reaction operating temperature while keeping the operating temperature of the microarray less than the reaction region operating temperature.

According to various embodiments, the method can comprise loading the binding sites with the reaction product. The loading can comprise dividing the reaction product into a plurality of aliquots, one aliquot for each binding site.

According to various embodiments, the method can comprise determining the presence or absence of different alleles. Each binding site can be adapted to react with a different allele. In various embodiments, more than one binding site can be adapted to detect the same allele. The presence or absence of an allele can then be related to its associated trait or lack thereof.

According to various embodiments, a system is provided that can comprise a platen, a fluid processing device holder disposed in the platen and adapted to hold at least one fluid processing device, and a detector adapted to detect a detectable characteristic from at least one of the binding sites. The fluid processing device can comprise a substrate that can comprise a surface and a fluid processing pathway at least partially formed in or on the surface. The fluid processing pathway can comprise a channel, a reaction region in fluid communication with the channel, a microarray in fluid communication with the channel, and optionally a deformable valve. The microarray can comprise a plurality of binding sites and each binding site can comprise at least one binding moiety.

According to various embodiments, the binding moiety can comprise a fluorescent emission or luminescence from at least one of the binding sites. The system can comprise an excitation source adapted to emit an excitation beam directed to at least one of the binding sites. The system can comprise a plurality of excitation sources. Each excitation source can be capable of emitting a respective excitation beam having a half-peak wavelength range substantially different than a half-peak wavelength range of other excitation sources in the plurality of excitation sources. Each excitation source can be adapted to direct the respective excitation beam to at least one of the binding sites. At least one excitation source of the plurality of excitation sources can emit a blue excitation beam. At least one excitation source of the plurality of excitation sources can emit a green excitation beam.

According to various embodiments, the platen can comprise an axis of rotation and the system can comprise a drive to spin the platen about the axis of rotation. The system can comprise a thermal cycler that can be adapted to heat the fluid processing device when the fluid processing device can be disposed in the fluid processing device holder. The thermal cycler can be adapted to heat a portion of the fluid processing device, for example, the reaction region, while keeping another portion of the fluid processing device cooler than the heated portion of the fluid processing device, for example, the microarray. U.S. patent application Ser. No. 10/926,915 filed Aug. 26, 2004, describes such a thermal cycler and is incorporated herein in its entirety by reference.

The system can comprise a fluid processing device that can be disposed in the fluid processing device holder. The system can comprise a cover that can be applied to the surface of the substrate to at least in-part cover a portion of the reaction region, the microarray, or both. The system can comprise a biological reagent disposed in the reaction region. The fluid processing device can comprise a biological reagent disposed in the microarray. The fluid processing device can comprise a plurality of fluid processing device holders.

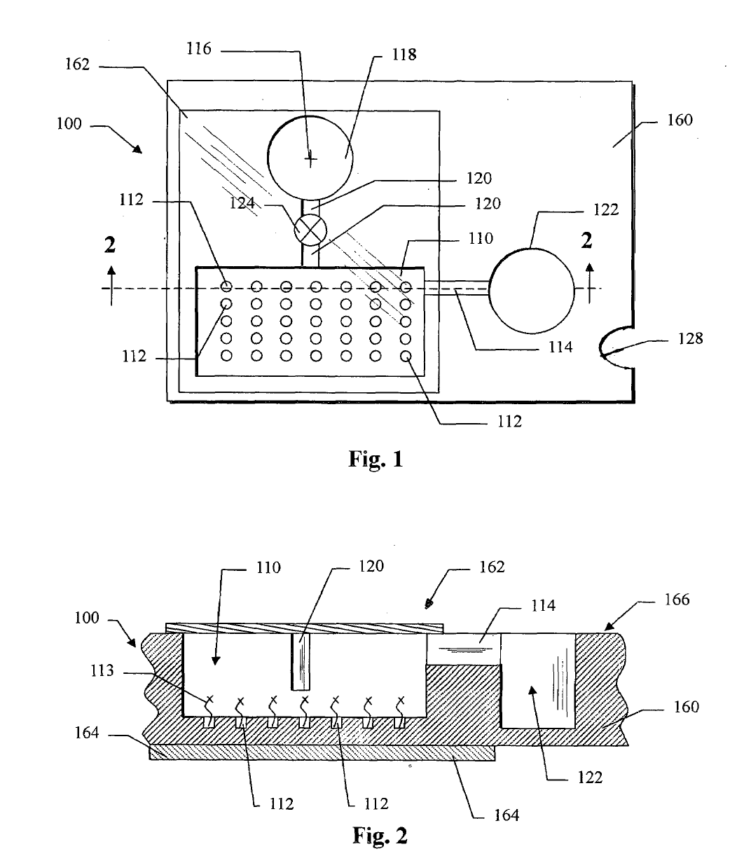

FIG. 1 illustrates an embodiment of fluid processing device 100 that can comprise substrate 160, reaction region 118, microarray 110, and waste chamber 122. Reaction region 118 can be in fluid communication with channel 120. Microarray 110 can comprise a plurality of addressable binding sites 112. Binding sites 112 can comprise wells, dips, depressions, through-holes, or the like, which can be adapted to hold a fluid. Binding sites 112 can be filled by capillary action. Binding sites 112 can be formed by coating a surface of each binding site with a hydrophilic coating. In various embodiments, binding sites 112 can be formed with a hydrophilic spray and/or a hydrophobic spray, each coated on a surface in a desired pattern to form an addressable pattern of binding sites 112. A fluid can be collected, gathered, pooled, bound, held, contained, or the like, in binding sites 112. The spots can comprise a binding moiety.

Microarray 110 can be in fluid communication with channel 120 that can have a valve or a flow control device 124 disposed therein or otherwise associated therewith. Microarray 110 can be in fluid communication with waste chamber 122, via channel 114. Substrate 160 can comprise an alignment notch 128 to align fluid processing device 100, when fluid processing device 100 is disposed in a system (not shown). Alignment notch 128 can be a partial or full cutout in substrate 160. Reaction region 118 can include an input port 116 for dispensing a sample or a reagent into the reaction chamber 118. Input port 116 can be a septum in cover 162. Cover 162 can be optically transparent.

Microarrays can be prepared as deemed appropriate by one of skill in the art. Nucleic acid sequences to be used in the microarray can be determined depending upon the desired application. Examples of microarrays, as well as their use, and hybridization assays can be found, for example, in U.S. Pat. No. 6,939,700 B2, issued Sep. 6, 2005, U.S. Pat. No. 6,916,447 B2, issued Jul. 12, 2005, U.S. Pat. No. 6,913,904 B2, issued Jul. 5, 2005, U.S. Pat. No. 6,887,664 B2, issued May 8, 2005, and U.S. Pat. No. 6,713,295 B2, issued Mar. 30, 2004, all of which are incorporated herein, by reference, in their entireties.

FIG. 2 is a side cross-sectional view of fluid processing device 100 taken along line 2-2 of FIG. 1. Substrate 160 can comprise surface 166. Microarray 110 can be formed in or on surface 166. Waste chamber 122 can be formed in or on surface 166. The reaction region 118 can be formed in or on the surface 166. The cover 162 can be disposed on the surface 166. The binding moiety 113 is enlarged for illustrative purposes. The binding moiety 113 can comprise a material disposed in or on a binding site. The binding sites 112 can each comprise a binding moiety 113. A thermal insulating layer 164 can be disposed on the substrate 160. The thermal insulating layer 164 can be disposed only on a portion of the substrate 160.

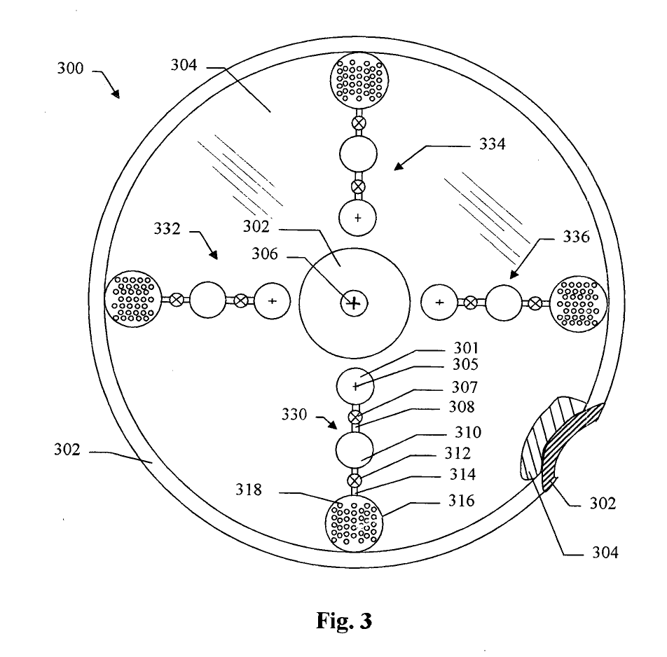

FIG. 3 illustrates an embodiment of fluidic device 300. Substrate 302 can comprise a surface and a plurality of fluid processing pathways 330, 332, 334, and 336 that can be formed therein or thereon. In various embodiments, greater or lesser members of processing pathways can be located on the fluidic device. For the sake of brevity, only fluid processing pathway 330 will be described. Fluid processing pathway 330 can comprise reaction region 301 and reaction region 310 in fluid communication with each other through channel 308. A fluid control device or valve 307 can be disposed in channel 308. Valve 307 can comprise a deformable valve, for example, a valve comprising an elastic cover layer and a less-elastic or non-elastic underlying material. Wherein simultaneous deformation of the elastic cover layer and the underlying material and subsequent elastic rebound or recovery of the elastic cover layer results in a through-passage channel formed between the elastic cover layer and the underlying material. The deformable valve can comprise an openable and reclosable deformable valve. Exemplary of such deformable valves are those described, for example, in U.S. patent application Ser. Nos. 10/336,274, filed on Jan. 3, 2003, 10/403,652, filed Mar. 31, 2003, 10/625,449, filed Jul. 23, 2003, and 10/403,640, filed Mar. 31, 2003, all of which are incorporated herein, in their entireties, by reference.

Reaction region 310 can be in fluid communication with channel 314. Channel 314 can be in fluid communication with microarray 316 comprising a plurality of binding sites 318. Valve 312 can be disposed in second channel 314. Cover 304 can be disposed on substrate 302 to form a seal over one or more of reaction region 301, reaction region 310, and microarray 316. Input port 305 can be disposed in cover 304 to allow access to reaction region 301. Additional ports can be formed in cover 304 to provide accesses other reaction regions. In various embodiments, the plurality of fluid processing pathways 330, 332, 334, and 336 can be arranged radially around a center of rotation 306, while in other embodiments, they can be arranged non-radially around a center of rotation 306. Reaction region 301 can be arranged radially inward from reaction region 310. Reaction region 310 can be arranged radially inward from microarray 316. Binding sites 318 can be formed in a honeycomb pattern or any other pattern deemed to maximize the use of a surface area of the microarray 316. Other patterns of binding sites can also be used and the present teachings are not intended to be limited to any particular pattern of binding sites.

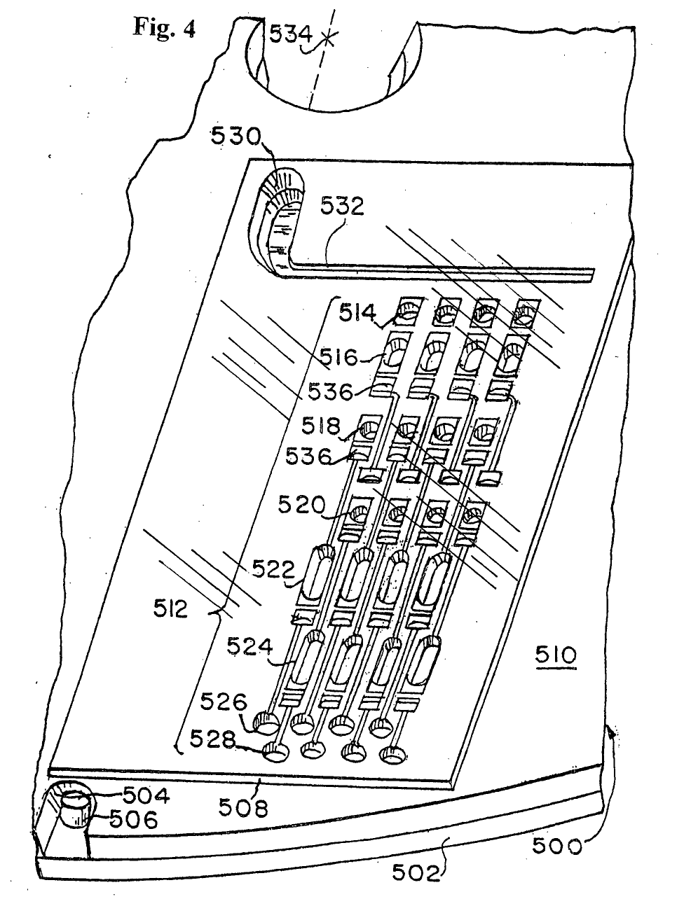

FIG. 4 is an enlarged, perspective view of fluid processing device 500, according to various embodiments, that can be used to manipulate fluids, for example, micro-sized fluid samples. Fluid processing device 500 can comprise substrate 510 that can comprise a plurality of fluid-containment features or fluid retainment regions formed therein or thereon, for example, a plurality of fluid retainment regions 514, 516, 518, 520, 522, 524. Fluid retainment regions can function to isolate or separate a volume of fluid. Greater or lesser numbers of such regions can be used as deemed appropriate. Fluid retainment regions 514, 516, 518, 520, 522, and 524 can each independently be formed in or on fluid processing device 500. Other fluid-containment features, for example, reservoirs, recesses, channels, vias, appendices, input wells and ports, output wells, purification columns, or valves, can be interconnected by valves, and can be included in or on the fluid processing device 500. In various embodiments, the valves can be deformable valves. The deformable valve can comprise a Zbig valve, for example, valve 536. Zbig valves are described, for example, in U.S. patent application Ser. No. 10/336,274, filed Jan. 3, 2003, which is incorporated herein, in its entirety, by reference. Valves can be arranged between any or all of fluid retainment regions 514, 516, 518, 520, 522, 524 to selectively control fluid communication between the fluid retainment regions. The deformable valve can be as described in U.S. patent application Ser. Nos. 10/808,228, filed Mar. 24, 2004, 10/808,229, filed Mar. 24, 2004, 10/336,274, filed Jan. 3, 2003, and 10/625,449, filed Jul. 23, 2003, which are incorporated herein, in their entireties, by reference.

According to various embodiments, substrate 510 of the fluid processing device 500 can be at least partially formed of a deformable material, for example, an inelastically deformable material, for example, a material that can change shape without rebound to its original shape. For example, a deforming blade can be used to deform substrate 510 to produce a deformation in the form of a channel between manifold 532 and each respective fluid region, for example, region 514.

Substrate 510 can include a single layer of material, a coated layer of material, a multi-layered material, or a combination thereof. Substrate 510 can be formed as a single layer. The single layer can be made of a non-brittle plastic material, for example, polycarbonate, a TOPAS material, or a plastic cyclic olefin copolymer material available from Ticona (Celanese AG), Summit, N.J., USA. The thermal conductivity of the TOPAS cyclic olefin copolymer can be about 0.16 Watt per meter Kelvin. Substrate 510 can be in the shape of a disk, a rectangle, a square, or any other shape. Substrate 510 can provide an operative surface for a thermal device to thermally contact the fluid processing device 500.

According to various embodiments, an elastically deformable cover sheet 508 can be adhered to at least one of the surfaces of substrate 510. Cover sheet 508 can be made of, for example, a plastic, an elastomer material, or other elastically deformable material. Fluid processing device 500 can include a central axis of rotation 534. Cover sheet 508 can provide an operative surface for a thermal device to thermally contact the fluid processing device 500. An input fluid-containment feature 530 can be fluidly connected to manifold 532 for the introduction of one or more fluids to fluid processing pathway 512 via a branch channel (not shown) or opening a valve (not shown). For example, one or more fluids can be introduced by piercing through the cover sheet 508 in the area of the input fluid-containment feature 530 and injecting one or more fluids into input fluid-containment feature 530. The fluid can be collected in output fluid retainment regions 526, 528 after being processed through fluid processing pathway 512. According to various embodiments, fluid processing pathway 512 can be arranged generally linearly. Other arrangements, however, can also be used. Fluid processing pathway 512 can be aligned radially or non-radially with respect to axis of rotation 534. In some embodiments non-radial alignments facilitate fluid transfer.

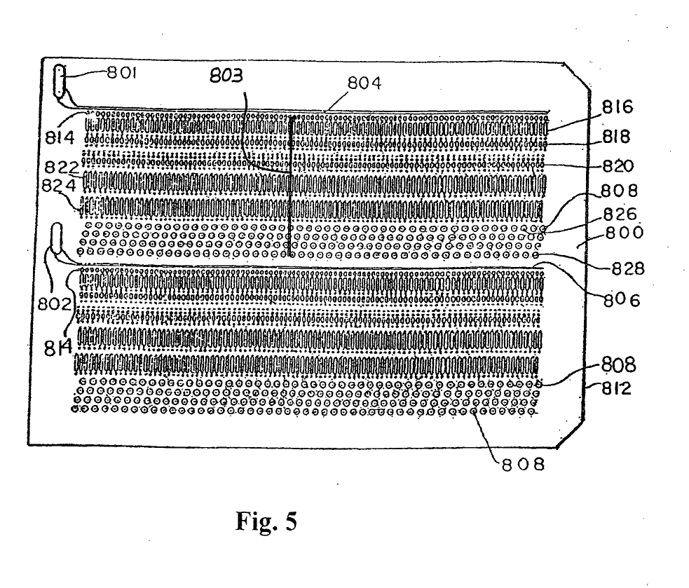

According to various embodiments, and as shown in FIG. 4 and FIG. 5, more than one fluid processing pathway 512 can be arranged side-by-side in or on substrate 510. A plurality of samples or a plurality of reactions on the same sample can be processed in fluid processing pathway 512. The processing can be serial or simultaneous. Processing can comprise a series of reactions performed one after another. For example, a sample preparation reaction can be carried out in one portion of the device to create a reaction product. The reaction product can then be flowed to another section of the device to perform an additional reaction. After this reaction, the new reaction product can be flowed to the microarray for analysis. In various embodiments, there can be 12, 24, 48, 96, 192, or 384 fluid processing pathways 512 arranged side-by-side on a substrate to form a set of fluid processing pathways on a single fluid processing device 500.

According to various embodiments, two or more sets of fluid processing pathways can be arranged on a single fluid processing device 500. One or more output fluid retainment regions 526, 528 can be provided in each fluid processing pathway 512. A plurality of binding sites (not shown) comprising detectable moieties can be disposed in output fluid retainment regions 526, 528.

Fluid retainment regions, for example, any or all of 514, 516, 518, 520, 522, 524 can be used, for example, to perform a nucleic acid amplification reaction (for example, a polymerase chain reaction), a nucleic acid purification reaction, a forward sequencing reaction, or reverse sequencing reactions, respectively. The reactions can be performed in the respective fluid retainment region of each fluid pathway 512, sequentially or simultaneously. After fluid processing device 500 has been used to perform the above set of reactions, output fluid retainment regions 526 and 528 can contain a forward sequencing reaction product and a reverse sequencing reaction product, respectively. The forward sequencing reaction product and the reverse sequencing reaction product can be loaded into the plurality of binding sites in the output fluid retainment regions 526 and 528. A plurality of single nucleotide polymorphisms (SNPs) or alleles can be detected.

According to various embodiments, fluid processing device 500 can be rotated through a central axis of rotation 534, to selectively force fluids between fluid retainment regions 514, 516, 518, 520, 522, 524 of fluid processing device 500, by way of applying a centripetal force. For example, by spinning the fluid processing device 500 around the central axis of rotation 534, a fluid can be selectively forced to move from at least input fluid-containment feature 530 to output fluid retainment regions 526, 528 along fluid processing pathway 512. In various embodiments, fluid flow can be controlled by the manipulation of valves 536.

According to various embodiments, platen 502, or a fluid processing device holder built-in platen 502, can be arranged to support and rotate fluid processing device 500 about the axis of rotation 534 of platen and/or holder 502. According to various embodiments, and as shown in FIG. 4, the axis of rotation of platen 502 can be coaxial with the central axis of rotation 534 of fluid processing device 500. An alignment notch 504 can be disposed in fluid processing device 500, to complement an alignment pin 506 in platen 502.

According to various embodiments, the movement of fluids within features of a fluid processing device can be provided by, for example, spinning, suctioning, or pneumatics.

According to various embodiments, the valve can be manipulated by, for example, a deformer, a flap, or a solenoid. Further information concerning manipulation of valves can be found in U.S. patent application Ser. No. 10/808,229 filed Mar. 24, 2004, which is incorporated herein, in its entirety, by reference.

According to various embodiments, FIG. 5 is a top view of an exemplary fluid processing device, comprising a fluid pathway similar to FIG. 4 comprising two input ports 801, 802, for distributing a fluid sample to respective flow distributors 804, 806, each flow distributor being in fluid communication with, or being adapted to be in interruptible communication with a plurality of fluid processing pathways. An exemplary pathway can comprise a PCR chamber to provide a PCR reaction product, a PCR reaction product purification chamber to purify the PCR reaction product, a forward sequencing chamber, and reverse sequencing chambers, and purification chambers to purify products from the sequencing chambers. Each chamber can be adapted to be in fluid communication with an input port and/or other chambers. As desired, during the process, the fluid communication can result form deforming deformable valves. Fluid processing device 800 can comprise any number of output ports deemed appropriate, for example, output ports 808. Each pathway 803 can include a PCR chamber 814, a PCR purification chamber 816, a flow restrictor, a vertical flow-splitter that leads to a forward sequencing chamber 818 and a reverse sequencing chamber 820, a forward sequencing product purification chamber 822, a reverse sequencing product purification chamber 824, a purified forward sequencing product output chamber 826, a purified reverse sequencing product output chamber 828, a plurality of opening and closing valves, or a combination thereof. The purified forward sequencing product output chamber 826 and the purified reverse sequencing product output chamber 822 can have binding sites disposed therein. Exemplary volumes for each of the chambers can be from about 0.01 microliters to about 100 microliters.

According to various embodiments, a reaction region can comprise various components or reagents for performing a polymerase chain reaction. The components and/or reagents in the reaction region can be mixed, dissolved, or contained in an aqueous solution. Components of a PCR mixture can comprise a buffer, KCl, dNTP's, primers, and a thermostable polymerase, for example, Thermus aquatus (Taq polymerase). The solution volume can depend on the reaction volume desired. For example, the aqueous solution can have a volume of from about 0.02 to about 200 μl. The aqueous solution can be a buffer. The buffer can have a pH of, for example, from about pH 8 to about pH 9 at room temperature. The aqueous buffer can contain, for example, about 0.05M potassium chloride (KCl). The dNTPs, for example, dATP, dTTP, dCTP, and dGTP, can have a concentration of, for example, from about 50 μM to about 100 μM. The primers can be oligonucleotide primers, for example, single-stranded DNA primers, single-stranded LNA primers, or single-stranded chimeric PNA primers “doped primers.” Examples of “doped primers” can be found in U.S. Pat. No. 6,887,986 B2, issued May 3, 2005, which is incorporated herein by reference in its entirety. The primers can be up to, for example, 15, 30, 45, 60, or more nucleotides long and can contain base sequences that are Watson-Crick complementary to sequences on one or both strands of target nucleic acid sequences. The primers can be present at a concentration of, for example, from about 50 to about 2000 nanomolars. To perform a sequencing reaction, at least some of the dNTPs can be dideoxynucleotide triphosphates (ddNTPs).

According to various embodiments, at least one of the reaction regions and the microarray can retain or contain one or more activating agents for isothermal nucleic acid sequence amplification or sequencing reaction.

According to various embodiments, at least one of the reaction regions or the microarray can retain or contain components necessary to perform a ligase chain reaction (LCR), rolling circle amplification, an oligonucleotide ligase assay (OLA), a ligase assay (LA), or an endonuclease reaction. The contents of the other reaction regions or the microarray can optionally contain various components or reagents for performing at least one of the above-mentioned reactions and/or assays. According to various embodiments, the region can contain other reagents. For example, the reaction region can retain an activating agent as described herein, and the microarray can retain at least one buffer, dNTPs, and at least one probe. The reaction region can retain a ligase, an endonuclease, or other enzyme(s). The components and/or reagents retained in the first chamber can be mixed, dissolved, or contained in an aqueous solution. The enzymes can be thermostable enzymes, than can be, for example, magnesium-dependent or magnesium-mediated enzymes.

According to various embodiments, the fluid processing device can be of the size, shape and general layout of a compact disk (CD). In other embodiments, the fluid processing device can be a card, for example, a rectangular fluid processing device card. The card can have one or more notches or other features that orient the card in another device, for example, in a card holder or on a rotating platen. The fluid processing device can be adapted to fit into a rotating platen or a fluid processing device holder. The platen can be attached to or connected with a mechanical device to spin the fluid processing device, heat the fluid processing device, agitate the fluid processing device, move the fluid processing device, or perform other physical manipulations of the fluid processing device, or combinations thereof.

According to various embodiments, the fluid processing device can be a monolithic structure. The fluid processing device can have at least two fluid retainment regions adapted to retain solutions or other reagents. The fluid processing device can have one or more valves that can be adapted to place at least two chambers in fluid communication. The fluid processing device can have a first side and a second side. Valves, chambers, fluid passages, or combinations thereof, can be located on the first side, the second side, or both sides of the fluid processing device. Valves or fluid passages can connect fluid retainment regions on the first side of fluid processing device with fluid retainment regions on the second side of the fluid processing device. The fluid retainment regions, valves, or fluid passages can have at least one side wall. The fluid retainment regions can be adapted to retain, contain, receive, restrain, archive, hold, and/or dispense reagents. The fluid retainment regions can be adapted to retain reactants during chemical reactions, for example, a polymerase chain reaction, a ligase chain reaction, an oligonucleotide ligase assay, an endonuclease assay, or a nucleic acid sequencing reaction. The fluid retainment regions can be adapted to perform filtration or purification of reagents or solutions.

One or more cover layers can cover the first and/or second sides of the fluid processing device. The cover layer can be optically clear. The cover layer can be thermally conductive or thermally insulating. In various embodiments, the cover layer can be elastically deformable or semi-elastically deformable. Adjacent sections of the cover layer can be made of one or more different materials.

According to various embodiments, there can be an intermediate layer between the cover layer and the fluid processing device. If the material of the intermediate layer is elastically deformable, it can be less elastically deformable (have less elasticity) than the material of the cover layer, or at least not be as quickly elastically rebounding as the material of the cover layer, whereby the cover layer is able to recover or rebound from deformation, more quickly than the intermediate wall material. Thus, if both the cover layer and the intermediate wall are elastically deformable but to different degrees, the cover layer can rebound from deformation more quickly than the intermediate wall material and a gap can be provided between the cover layer and the intermediate layer, that can function as an opening for a fluid communication.

Examples of fluid processing device features and systems for spinning, heating, cooling, and otherwise processing fluid processing devices, that can be useful in or with the fluid processing devices, can be found in U.S. patent application Ser. Nos. 10/336,274, filed Jan. 3, 2003, 60/398,851, filed Jul. 26, 2002, 60/399,548, filed Jul. 30, 2002, 10/336,706, filed Jan. 3, 2003, 60/398,777, filed Jul. 26, 2002, 10/403,652, filed Mar. 31, 2002, 60/398,946, filed Jul. 26, 2002, 10/336,330, filed Jan. 3, 2003, and 10/403,640, filed Mar. 31, 2003, which are incorporated herein in their entireties by reference.

According to various embodiments, the fluid retainment regions can be preloaded with reagents or reactants. For example, the reaction region or the microarray can be preloaded with a buffer solution, Taq polymerase, a chelating agent, a ligase, an endonuclease, dNTPs, one or more salt, such as KCl, one or more primer, one or more probe, or combinations thereof. A user can load a sample containing DNA into a third chamber that can be in fluid communication with the reaction region and/or microarray by one or more valves. The reaction region can contain the necessary components of a single-tube assay, described, for example, in PCT International Patent Application No. PCT/US03/02238, filed on Jan. 27, 2003, which is incorporated herein in its entirety by reference. In various embodiments, a user can load additional solutions or reactants into the already preloaded regions.

According to various embodiments, the valve can be a pressure-sensitive one-way valve or a single use valve. The valve can be an elastically deformable barrier. For example, the valve can be a deformable barrier where one or more sidewalls of the valve can be deformed to close the valve. Alternatively, or additionally, a barrier can be deformed to open the valve. The valve can be a Zbig valve. The valve can include an elastic material. The valve can be as described for example, in U.S. patent application Ser. Nos. 60/398,851, filed Jul. 26, 2002, 60/399,548, filed Jul. 30, 2002, 10/336,706, filed Jan. 3, 2003, 60/398,777, filed Jul. 26, 2002, 10/403,652, filed Mar. 31, 2002, 60/398,946, filed Jul. 26, 2002, 10/336,330, filed Jan. 3, 2003, and 10/403,640, filed Mar. 31, 2003, which are incorporated herein in their entireties by reference.

The reaction region can be loaded with one or more reactants necessary to perform a nucleic acid polymerase chain reaction. The reaction region and/or microarray can include buffers, salts, chelating agents, polymerases, other enzymes, dNTPs, primers, and sample nucleic acid sequence, DNA, or DNA fragment. According to various embodiments, the contents of the chamber with such components can be heated to a temperature sufficient to denature a majority of a sample. For example, the temperature can rapidly be heated from room temperature to a temperature from about 90° C. to about 10° C. According to various embodiments, a valve, for example, an elastically deformable valve between the reaction region and the microarray, can be opened while the contents of one of the reaction regions is heated, so that the reaction region and the microarray become in fluid communication with each other. The fluid processing device, for example, a fluid processing card device, containing the reaction region and the microarray, can be mounted on a platen that is connected to a mechanical device. The platen can be rotated to move the contents of the reaction region into the microarray using centripetal force. While rotating or spinning, heat can continuously applied to the reaction region and the microarray.

The contents of the reaction region and the microarray can be combined and/or mixed using centripetal force by rotating the fluid processing device, to initiate a reaction of the various components, for example, a polymerase chain reaction. The contents of the reaction region and the microarray can be cooled, heated, or combinations thereof, between the ligase reaction and the polymerase chain reaction. For example, the temperature level can be permissive to initiate, promote, maintain, or activate a chemical reaction for use with real-time monitoring of a polymerase chain reaction. Room temperature can be sufficient to initiate, promote, or maintain a flap endonuclease (FEN) reaction according to various embodiments.

The sample components or analytes to be measured can be labeled to facilitate sensitive and accurate detection. Labels may be direct labels which themselves are detectable or indirect labels which are detectable in combination with other agents. Exemplary direct labels include but are not limited to fluorophores, chromophores, spin-labels, chemiluminescent labels, dioxetane-producing moieties, radioisotopes, and the like. Detectable moieties or analytes in a sample to be measured can be labeled to facilitate accurate and sensitive detection. Labels can be direct labels which themselves are detectable, or indirect labels which are detectable in combination with other agents. Exemplary direct labels include, for example, fluorophores, chromophores, spin-labels, chemiluminescent labels, dioxetane-producing moieties, radio isotopes, or nanoprobes. Exemplary indirect labels include enzymes which catalyze a signal-producing event, and ligands such as an antigen or biotin which can bind specifically with high affinity to a detectable anti-ligand, such as a labeled antibody or avidin. Many references on labeling molecules of interest, such as DNA, proteins, polysaccharides, and the like, are available. Exemplary references include Matthews et al. (1988), Haugland (1992), Keller and Manak (1993), Eckstein (1991), Fung et al.; Hobbs et al., Lee et al., Menchen et al., Bergot et al., Rosenblum et al. (1997), and Jackson (WO 91/05256).

The present teachings relate to the foregoing and other embodiments as will be apparent to those skilled in the art from consideration of the present specification and practice of the present teachings disclosed herein. It is intended that the present teachings be considered as exemplary only.

What is claimed is:

1. A fluid processing device comprising:
a substrate comprising a surface; and
at least one fluid processing pathway at least partially formed in or on the surface, the fluid processing pathway comprising,
a channel comprising a first end and a second end,
a reaction region in fluid communication with the first end,
a deformable valve adapted to open and provide fluid communication through the channel,
a fluid retainment region in fluid communication with the second end, and
a microarray disposed in the fluid retainment region, the microarray comprising a plurality of binding sites, each binding site comprising an immobilized moiety,
wherein at least two of the binding moieties are different from one another.

2. The fluid processing device of claim 1, wherein the reaction region has a volume of from about 1 μl to about 100 μl.

3. The fluid processing device of claim 3, wherein the at least one reagent comprises a reagent for a nucleic acid amplification reaction.

4. The fluid processing device of claim 1, wherein the binding moiety of one of the binding sites comprises an oligonucleotide.

5. The fluid processing device of claim 1, wherein the binding moiety comprises at least one of a 5′-exonuclease probe, a stem-loop beacon probe, or a stemless beacon probe.

6. The fluid processing device of claim 1, wherein the at least one fluid processing pathway comprises a plurality of fluid processing pathways, each fluid processing pathway comprising a respective reaction chamber.

7. The fluid processing device of claim 1, wherein binding sites have a density of at least one binding site per 10 μm².

8. A method comprising:
loading a sample into a reaction region of a fluid processing device;
reacting the sample in the reaction region to form a reaction product;
opening a deformable valve along a fluid communication leading away from the reaction region;
moving the reaction product from the reaction region through the fluid communication and into a hybridization array; and
reacting the reaction product with binding moieties of the microarray.

9. The method of claim 8, wherein the moving of the reaction product comprises spinning the fluid processing device to centrifugally propel the reaction product.

10. The method of claim 8, further comprising detecting a detectable characteristic resulting from the reaction of the reaction product with the binding moieties.

11. The method of claim 10, wherein detecting comprises detecting luminescence or fluorescence emissions from at least one binding site of a plurality of binding sites.

12. The method of claim 11, wherein detecting comprises illuminating at least one the plurality of binding sites with at least one excitation beam.

13. The method of claim 8, wherein the sample comprises at least one oligonucleotide.

14. The method of claim 8, wherein the reaction product comprises at least one of a nucleotide amplification reaction product and a sequencing reaction product.

15. The method of claim 8, further comprising purifying the reaction product prior to moving the reaction product into the microarray.

16. A system, comprising:
a platen;
a fluid processing device holder disposed in the platen and adapted to hold at least one fluid processing device, comprising,
a substrate comprising a surface; and
at least one fluid processing pathway at least partially formed in or on the surface, the fluid processing pathway comprising;
a channel comprising a first end and a second end,
a reaction region in fluid communication with the first end,
a deformable valve adapted to open and provide fluid communication through the channel,
a fluid retainment region in fluid communication with the second end, and
a microarray disposed in the fluid retainment region, the microarray comprising a plurality of binding sites, each binding site comprising an binding moiety,
wherein at least two of the binding moieties are different from one another; and
a detector adapted to detect a detectable characteristic from at least one of the binding sites.

17. The system of claim 16, wherein the platen comprises an axis of rotation and the system further comprises a drive to spin the platen about the axis of rotation.

18. The system of claim 16, wherein the detectable characteristic comprises a fluorescent emission from at least one of the binding sites.

19. The system of claim 41, further comprising an excitation source adapted to emit an excitation beam directed to at least one of the binding sites.

20. The system of claim 16, further comprising a plurality of excitation sources, each excitation source adapted to emit a respective excitation beam having a half-peak wavelength range substantially different than a half-peak wavelength range of other excitation sources in the plurality of excitation sources, and each excitation source is adapted to direct the respective excitation beam to at least one of the binding sites.
